(19) United States
(12) Patent Application Publication
(10) Pub. No.: US 2023/0175041 A1
(43) Pub. Date: Jun. 8, 2023

(54) DYNAMIC FRET-BASED SINGLE-MOLECULE SENSOR FOR ULTRASENSITIVE DETECTION OF NUCLEIC ACIDS

(71) Applicant: VIRGINIA COMMONWEALTH UNIVERSITY, Richmond, VA (US)

(72) Inventors: Soma DHAKAL, Richmond, VA (US); Kumar SAPKOTA, Richmond, VA (US)

(21) Appl. No.: 17/904,741

(22) PCT Filed: Feb. 25, 2021
(86) PCT No.: PCT/US2021/019553

§ 371 Date: Aug. 22, 2022

Related U.S. Application Data
(60) Provisional application No. 62/981,338, filed on Feb. 25, 2020.

(51) Int. Cl. C12Q 1/6818; G01N 15/06

(52) U.S. Cl. CPC C12Q 1/6818; G01N 15/0612; G01N 2015/0065

(57) ABSTRACT

Fluorescence resonance energy transfer (FRET)-based nucleic acid sensors and methods of their use for detecting nucleic acids are provided. The sensors are highly sensitive and detect nucleic acids at the femtomolar (fM) level, without the need for labeling and amplification.

### CROSS-REFERENCE TO RELATED APPLICATIONS

This application claims benefit of United States provisional pat. application 62/981,338 filed Feb. 25, 2020.

### BACKGROUND OF THE INVENTION

### Field of the Invention

The invention generally relates to improved nucleic acid sensors and methods of their use for high-confidence detection of nucleic acids. In particular, the invention provides a unique fluorescence resonance energy transfer (FRET)-based dynamic sensor that is highly specific to target and can detect even trace amounts (low attomoles) of nucleic acids with high confidence.

### Description of Related Art

There are several technologies available for nucleic acid detection and analysis such as hybridization, strand displacement, and enzymatic and non-enzymatic amplification assays. These techniques employ either single-molecule or ensemble approaches including optical, electrochemical, and colorimetric assays. Although hybridization-based assays offer a simple and fast analysis of nucleic acid targets, they typically exhibit poor specificity, e.g. between the target with perfect complementarity and a sequence with a point mutation. Therefore, any sensing approach that is sensitive down to a single-nucleotide mismatch can be very useful to ensure specificity of diagnosis. Although enzymatic amplification approaches such as polymerase chain reaction (PCR), reverse transcriptase PCR (RT-PCR), and digital PCR are simple and highly sensitive, they rely on target amplification and are susceptible to false negatives/positives. This is problematic because false negatives run the risk of instilling a false sense of security and false positives may result in an unnecessary panic. Therefore, novel single molecule and ensemble techniques with improved sensitivity and specificity are continuously emerging. Nonetheless, most of these methods are rather complicated and have limited applications. For example, techniques including synthetic nanopores, barcodes, and force-based approaches are limited by the need for precise and sophisticated engineering. Also, most of these methods require targets to be labeled, modified, or amplified to enable detection.

There is a need to develop simpler, more accurate sensors for nucleic acid detection and analysis.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Disclosed herein is a biomolecule sensor based on a 4-way DNA junction design comprising a donor and an acceptor fluorophore-pair, and changes in FRET signaling between the fluorophore-pair. Prior to use, the sensor is in the form of an incomplete 4-way DNA junction comprising a single strand DNA binding site that is complementary to a targeted nucleic acid sequence. In the incomplete 4-way DNA junction conformation(s), the fluorophore-pair emits a relatively static and readily detectable mid-FRET signal.

However, when the targeted nucleic acid sequence hybridizes to the single strand binding site of the sensor, forming a complete 4-way DNA junction, the conformation of the sensor changes and the fluorophore-pair undergoes continuous, readily detectable dynamic switching between a low- and high-FRET state. The sensor advantageously has a detection limit down to low femtomolar (fM) concentrations of DNA without the need for target amplification and is highly effective in discriminating, for example, single nucleotide polymorphisms (SNPs). Given the generic hybridization-based detection platform of the sensor, it has the potential to detect a wide range of nucleic acid sequences, enabling early diagnosis of diseases and screening of genetic disorders.

It is an object of this invention to provide a sensor, comprising a substrate, and an incomplete 4-way DNA junction immobilized on the substrate; wherein the incomplete 4-way DNA junction comprises a first arm of double-stranded(ds) DNA; a second arm of dsDNA; a third arm of single-stranded DNA; a fourth arm of single-stranded DNA; a fluorescence resonance energy transfer (FRET) donor; and a FRET acceptor, wherein the ssDNA of the third are and the fourth arm form a single strand binding site complementary to a targeted nucleic acid sequence; and wherein one of the FRET donor and the FRET acceptor is attached to dsDNA of the first arm and the other of the FRET donor and the FRET acceptor is attached to dsDNA of the second arm of the sensor.

In some aspects, the FRET donor and the FRET acceptor exhibit a detectable static mid-FRET state when the targeted nucleic acid sequence is not bound to the sensor; and the FRET donor and the FRET acceptor undergo detectable continuous dynamic switching between a low- FRET state and high-FRET state when the targeted nucleic acid sequence is bound to the sensor. In additional aspects, the incomplete 4-way DNA junction is converted to a complete 4-way DNA junction when the targeted nucleic acid sequence is bound to the sensor. In additional aspects, the incomplete 4-way DNA junction is immobilized on the substrate via a biotin/streptavidin interaction.

Also provided is a method of detecting a targeted nucleic acid sequence in a serum sample, comprising i) contacting the serum sample with the sensor of claim 1; And ii) detecting continuous dynamic switching of the FRET donor and the FRET acceptor between low-FRET and high-FRET levels, wherein detection of continuous dynamic switching indicates that the targeted nucleic acid sequence is bound to the sensor. In some aspects, the targeted nucleic acid sequence comprises at least one mutation. In further aspects, the at least one mutation is a point mutation, a deletion or an insertion. In additional aspects, the point mutation is a single nucleotide polymorphism (SNP).

### BRIEF DESCRIPTION OF THE DRAWINGS

FIG. 1. Working principle of the sensor. The sensor is composed of synthetic DNA strands, labeled with a Cy3 and a Cy5 fluorophore. The DNA construct exhibits a relatively steady FRET efficiency in the absence of a target. However, binding of the target forms a four-way structure resulting in a dynamic switching between a high (FRET₁) and a low (FRET₂) FRET states. FRET represents FRET efficiency.

FIG. 2. Typical single-molecule traces in the absence of target. Typical intensity-time (left) and corresponding FRET-time traces (right). Five representative molecules are shown. The molecules exhibited static fluorescence intensities of Cy3 and Cy5 in the absence of target and a static FRET level of ~0.5 was observed in the absence of target DNA. All experiments were done at room temperature (23° C.). FRET represents FRET efficiency.

FIG. 3. Detection of a target sequence (p53 tumor suppressor gene) using single-molecule FRET. Typical intensity-time (left) and corresponding FRET-time traces. Five representative molecules are shown. The molecules exhibited dynamic and anti-correlated fluorescence intensities of Cy3 and Cy5. Such dynamic FRET-time traces with FRET levels of ~0.3 and ~0.7 were obtained only in the presence of target DNA. All of the experiments were performed at room temperature (23° C.). FRET represents FRET efficiency.

FIG. 4. Determination of the limit of detection (LOD). Calibration curve was obtained by plotting the number of dynamic molecules (≈ target-bound molecules) as a function of target concentration. Inset shows the linear range of the calibration curve with a LOD of 50 fM. Considering our experimental volume of ~100 µL, this LOD is equivalent to 5 attomoles of DNA target. (1 attomoles = 1 × 10⁻¹⁸ moles). The results for both the original and revised designs are shown. The percentage of dynamic molecules were determined from more than 150 single molecules at each concentration. The error bars represent standard deviation from three groups of independent movie files.

FIGS. 5A and 5B. Specificity of sensors and their compatibility in serum. A. Specificity test of the sensor using 100 pM target/mutants. While the target is perfectly complementary, mutants have one or two mismatched nucleotides (bolded and underlined). We found that 70% of molecules were dynamic in the presence of 100 pM target, while a negligible fraction of molecules showed dynamic behavior in the presence of 100 pM mutant, thus demonstrating a high specificity of our approach. Sequences depicted in FIG. 5A are: target (SEQ ID NO: 3); mutant 1 (SEQ ID NO: 7); mutant 2 (SEQ ID NO: 8); and mutant 3 (SEQ ID NO: 9). B. Sensors behave similarly in 1x Tris HCI and 10% human serum. Notice the zero background in the absence of the target.

FIG. 6. The 4-way sensor design with corresponding strand names (A to F) listed in Table 1. Strand A = SEQ ID NO: 1; Strand B = SEQ ID NO: 2; Strand C = SEQ ID NO: 3; Strand D = SEQ ID NO: 4; Strand E = SEQ ID NO: 5; Strand F = SEQ ID NO: 6; Biotin, Cy3 and Cy5 labels as well as target have been identified.

FIG. 7. Typical single molecule traces in the presence of mutant 1 (Mut1). Typical intensity-time (left) and corresponding FRET traces (right). Five representative molecules are shown. The molecules exhibited static fluorescence intensities of Cy3 and Cy5. A static FRET level of ~0.5 was observed in the absence of target DNA. All experiments were done at room temperature (23° C.).

FIG. 8. Schematic depiction of an exemplary sensor.

FIG. 9. Alternative sensor design with connected strands 10 and 20 as well as connected strands 30 and 40.

FIG. 10. Alternative sensor design with a slightly longer (extended by about 2 to 4 base pairs) arm 3.

### DETAILED DESCRIPTION

Disclosed herein is a simple background-free fluorescence resonance energy transfer (FRET)-based sensor for ultrasensitive detection of nucleic acid sequences of interest. Unlike conventional fluorescence sensors which either require a complex design or signal amplification steps, or the use of additional materials such as enzymes or nanocomposites, the present sensor is simple and allows single-step detection of e.g. DNA biomarkers without the need for signal amplification. The sensor design is unique as the detection is based on dynamic FRET signaling, is less susceptible to background noise than other detection methods and can easily discriminate between targets with only 1 or 2 nucleotide mutations. The disclosed sensor achieves low femtomolar (10⁻¹⁵ M) detection limits, even without amplification. In addition, the sensor demonstrated zero background signaling so it allows high-confidence detection.

### Definitions

Fluorescence resonance energy transfer (FRET) (Förster resonance energy transfer (FRET), resonance energy transfer (RET) or electronic energy transfer (EET)) is a mechanism describing energy transfer between two light-sensitive molecules (chromophores). A donor chromophore, initially in an electronic excited state due to absorption of energy of a wavelength within its absorption spectrum, may transfer energy to an acceptor chromophore through nonradiative dipole-dipole coupling. The transfer of energy between donor and acceptor is a distance-dependent process and occurs without emission of a photon. Measurements of FRET efficiency can be used to determine if two fluorophores are within a certain distance of each other, typically less than 10 nm.

A 4-way DNA junction (supramolecular 4-way DNA junction, “Holliday junction”) is a branched nucleic acid structure that contains four double-stranded arms when complete.

### Overview of the Sensor

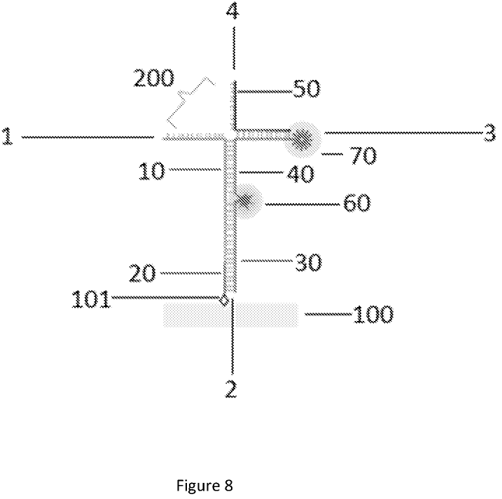

The present sensor comprises DNA strands immobilized on a substrate which initially form an incomplete 4-way DNA junction in that two adjacent arms of the 4-arm sensor are comprised of double strand (ds) base-paired DNA, while the two remaining arms of the sensor (also adjacent to each other) comprise single-stranded DNA that is not base-paired (see FIG. 8). The unpaired, single strand DNA of the sensor has a nucleotide sequence that is complementary to a nucleic acid of interest, such as a nucleic acid sequence that is targeted for detection. The single strand portion thus forms a binding site for the targeted nucleic acid sequence.

The ds portion of the sensor comprises a donor and acceptor fluorescence pair. Generally, the donor is attached to one ds arm of the junction and the acceptor is attached to another ds arm. Detection of the targeted nucleic acid sequence of interest depends on detecting the change in FRET signaling that occurs between the donor and acceptor that occurs when the sensor transitions from an incomplete 4-way junction to a complete 4-way junction upon binding of the targeted sequence. Generally, a static FRET signal is observed in the absence of a bound target sequence. However, the arms of a complete 4-way DNA junction are somewhat flexible and may adopt defined conformations which depend on e.g. buffer salt concentrations and the sequence of nucleobases closest to the junction. Spontaneous interconversion of the arms of the complete 4-way DNA junction between conformations cause variations (fluctuations) in the distance between the acceptor and donor fluorophore, resulting in a characteristic dynamic “high-low” FRET signal in the single-molecule FRET traces. This dynamic signal is readily distinguishable from the relatively static signal obtained when no target is bound to the sensor.

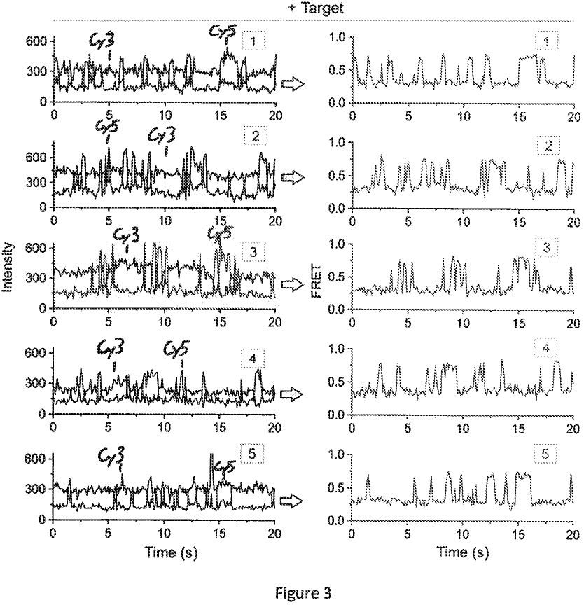

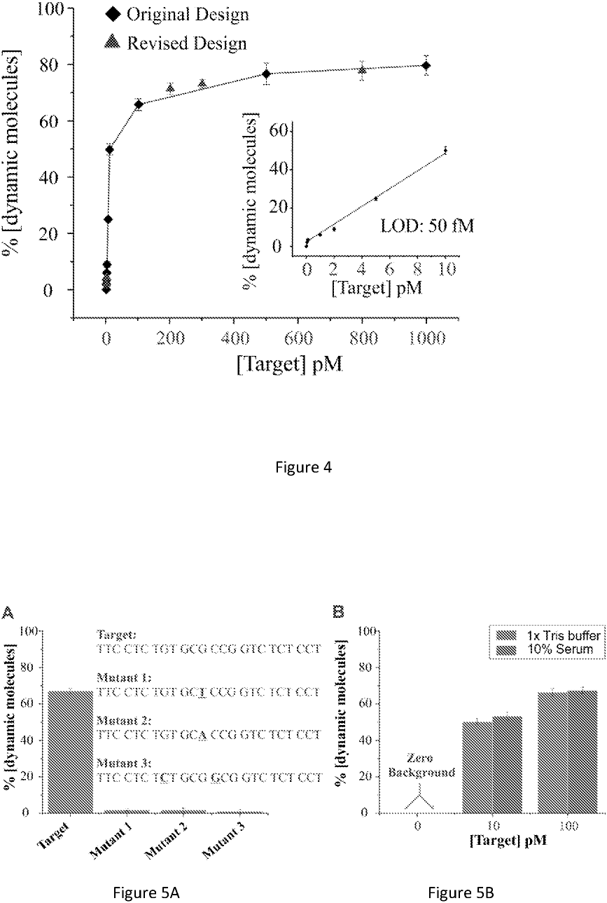

By a “relatively static FRET signal” we mean that the signal is not a characteristic dynamic “high-low” FRET signal but rather exhibits a relatively steady FRET efficiency. A “relatively static signal” means that the signal is relatively flat without any definite dynamic pattern and may be referred to as a “static mid-FRET state”. Generally static FRET-time traces have very narrow FRET values of from about 0.4 to about 0.6, such as about ~0.5 as shown in FIG. 3, in contrast to dynamic FRET signals, which vary from about 0.3 to about 0.7, such as about 0.3, 0.4, 0.5, 0.6 or 0.7, as shown in FIG. 4.

### Design and Detailed Structure of the Sensor

To detect a sequence of interest, it is necessary to know its exact nucleotide sequence and to synthesize a strand of DNA complementary thereto for use in constructing the sensor. The complementary strand functions as the single stranded portion of the incomplete 4-way DNA junction. In addition, other single strand DNA molecules are synthesized which, once annealed (hybridized, base-paired) to each other, will make up the ds portion of the 4-way DNA junction. The strands are designed so as to be complementary when assembled in a solution or on a substrate.

Donor and acceptor molecules of a donor and acceptor fluorescence pair are attached to the double-strand portion of the junction. The donor is attached to one ds arm of the incomplete (target is not bound) sensor and the acceptor is attached to the other ds arm of the incomplete sensor. The efficiency of FRET is dependent on the inverse sixth power of the intermolecular separation and is thus sensitive to changes in molecular proximity. Thus, the donor and acceptor molecules are positioned in the incomplete sensor with a distance between them over which a FRET signal is not generated or is generated at a very low and static (consistent) level. However, when a target nucleic acid that is a match for the ss DNA portion of the sensor binds to the sensor, the relative positions of the four arms of the sensor change as it adopts a complete 4-way junction conformation in which all nucleotides are base-paired. In the complete 4-way junction conformation, the donor and acceptor dye molecules are, at least on some conformations, brought into closer proximity, close enough to permit energy transfer from the donor to acceptor molecules, and thereby generate a FRET signal. The FRET signal is dynamic, as described above. Detection of a dynamic FRET signal thus indicates that a complementary nucleic acid, i.e. the target nucleic acid, was present in the sample and has bound to the sensor.

In one exemplary aspect, as is shown in FIG. 8, five separate DNA strands make up the incomplete 4-way DNA junction, as follows:
1. First strand 10 comprises a first portion comprising nucleotides that are complementary to a first portion of the target sequence (e.g. to about 50% of the nucleotides of the target sequence, to form (incomplete, single strand) arm 1 of the structure, which is one section of target binding site 200; and a second portion comprising nts that are complementary to and, together with another strand or other strands or portions of other strands of the structure, will form part of one ds arm (arm 2) of the junction. The first, single strand portion of first strand 10 is typically from about 5-15 nts long and preferably from about 9 to about 12 nts long e.g. about 5, 6, 7, 8, 9, 10, 11, 12, 13, 14, or 15 nt long; and the second portion of first strand 10 is typically from about 30 to about 60 nts long, and preferably from about 40 to about 50 nts long, such as about 30, 35, 40, 45, 50, 55 or 60 nts.
2. Second strand 20 comprises nts that are complementary to and, together with another strand or other strands or portions of other strands of the structure, form part of one ds arm of the junction (arm 2), and comprises e.g. at the 5′ end of the strand, molecular binding means 101 that permits immobilization of the structure on substrate 100 via arm 2. (FIG. 8 depicts the substrate and incomplete 4-way DNA junction after immobilization.) In some aspects, he substrate 100 is modified with, for example, biotinylated bovine serum albumin (BSA, or via another biotinylated molecule, protein, peptide, etc.) that attaches nonspecifically on the substrate surface to allow binding of streptavidin. Molecular binding means 101, typically biotin, that is covalently attached to the 5′ end of strand 20 binds to streptavidin that is attached to the substrate 100 via e.g. biotinylated BSA. Other molecular binding means may also be used, for example, thiol (S-H). Alternatively, amines (NH2) covalently linked at the 3′ or 5′ end of the DNA strand can be used to covalently attach the structure to the specific functional group (such as carboxyl, aldehyde, sulfonic, epoxy isothiocyanate group) introduced on the substrate 100 (see Rashid and Yusof, Science Direct, Volume 16, November 2017, Pages 19-31). Second strand 20 is typically from about 10 to 50 nt long and preferably from about 20 to about 40 nts long, such as about 10, 15, 20, 25, 30, 35 or 40 nts long.
3. Third strand 30 comprises nts that are complementary to and, together with another strand or other strands or portions of other strands of the structure, form part of one ds arm of the junction (arm 2). In addition, as shown in this exemplary figure, third strand 30 comprises FRET donor 60 covalently attached thereto at the 5′ end of the strand. It is noted that the positions of FRET donor 60 and FRET acceptor 70, as depicted in FIG. 8, can be reversed, as long as the sensor is operable and functions as described herein. Third strand 30 is typically from about 30 to 90 nts long and preferably from about 40 to about 80 nts long, such as about 30, 35, 40, 45, 50, 55, 60, 65, 70, 75 80, 85 or 90 nts long.
4. Fourth strand 40 comprises nts that are complementary to and, together with another strand or other strands or portions of other strands of the structure, form part of two ds arm of the junction (arm 2 and arm 3). In addition, as shown in this exemplary figure, fourth strand 40 comprises FRET acceptor 70 covalently attached thereto at the 5′ end of the strand. As noted above, the positions of FRET donor 60 and FRET acceptor 70, as depicted in FIG. 8, can be reversed, as long as the sensor is operable and functions as described herein. Fourth strand 40 is typically from about 10 to 40 nts long and preferably from about 20 to about 30 nts long, such as about 10, 12, 14, 16, 18, 20, 22, 24, 26, 28, 30, 32, 34, 36, 38, or 40 nts.
5. Fifth strand 50 comprises a first portion comprising nucleotides that are complementary to a second portion of the target sequence (e.g. to about 50% of the nucleotides of the target sequence, those to which first strand 10 are not complementary) thereby forming (incomplete, single strand) arm 4 of the sensor, which, together with arm 1, forms target binding site 200; and a second portion comprising nts that are complementary to and, together with another strand or other strands or portions of other strands of the structure, form part of one ds arm of the junction, e.g. arm 3. The first, single strand portion of fifth strand 50 is typically from about 5-15 nts long, preferably from about 9 to about 12 nts , such as about 5, 6, 7, 8, 9, 10, 11, 12, 13, 14, or 15 nts long; and the second portion of fifth strand 50 is typically from about 5-15 nts long, preferably from about 11 to about 13 nts long, such as about 5, 6, 7, 8, 9, 10, 11, 12, 13, 14, or 15 nts long.

Altogether, the binding site of the sensor typically comprises from about 15 to 30 nts, preferably about 18 to 24 nts, such as about 15, 16, 17, 18, 19, 20, 21, 22, 23, 24, 25, 26, 27, 28, 29, or 30 nts.

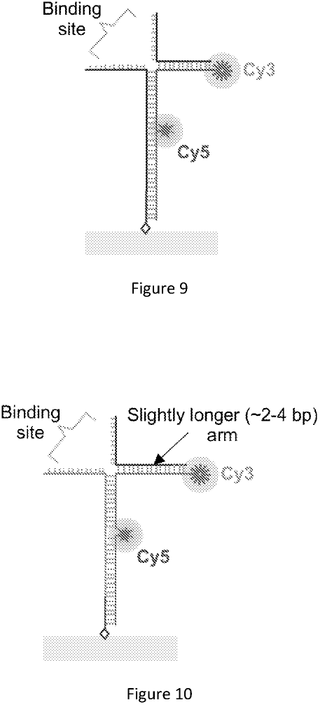

In other exemplary sensors, the nucleic acid compositions of the sensor may be designed and/or arranged differently, as long as an incomplete 4-way DNA junction is formed prior to binding of a targeted nucleic acid sequence. For example, the nts of first strand 10 and second strand 20 may be joined to form one strand (FIG. 9), one end of which comprises nts which are complementary to the target sequence and the other of which comprises a means for attaching the incomplete 4-way junction to the substrate. Alternatively, the nts of strands 30 and 40 may be joined to form a single strand to which the donor and acceptor moieties are attached at a suitable distance. Other such sensor designs may also be used, as long as the resulting sensor forms an incomplete 4-way DNA junction comprising two arms that form a single strand binding site for a target sequence, and which comprises a FRET donor and acceptor positioned so as to send a static signal prior to binding of the target sequence, and a detectable dynamic low-high signal when the target sequence is bound to the sensor. In other exemplary designs, the arm 3 of the sensor can be extended by about 2-4 base pairs (e.g. about 1, 2, 3, 4, or 5 base pairs) to increase thermodynamic stability of the arm (FIG. 10).

To generate a FRET signal, the donor and acceptor of the fluorophore-pair must be in proximity, for example, within about 10-100 Å in the complete 4-way junction. In addition, for FRET to occur, the donor and acceptor transition dipole orientations must be approximately parallel. Thus, in the incomplete junction, the donor and acceptor fluorophores are generally separated by a certain distance (e.g. from about 30 to about 50 Å, such as about 30, 35, 40, 45 or 50 Å), and the change from incomplete 4-way junction in the absence of target to a complete 4-way junction (which is inherently dynamic) in the presence of target brings them into the signal-generating range of distance, generally intermittently, thereby generating a readily detectable dynamic signal.

Fluorophore-pairs that may be used in the present FRET sensors include but are not limited to various fluorescent dyes such as a pair of cyanine dyes that make a FRET pair (used in this invention), cyan fluorescent protein (CFP) and yellow fluorescent protein (YFP) pair (color variants of green fluorescent protein, GFP); Fluorescein and Tetramethylrhodamine; and IAEDANS and Fluorescein.

Further, the incomplete 4-way DNA junction is immobilized on a substrate. Examples of substrates on which the incomplete 4-way DNA junction can be immobilized include but are not limited to: surfaces made of or comprising e.g. glass, quartz, various metals, etc., and/or composites of these. The form of the substrate can be any that is suitable for use in the methods described herein, e.g. flat surfaces such as microscope slides, chips for use in automated processes, beads (e.g. a metal bead, a gold bead, a polystyrene bead, etc.) tubes (e.g. the interior of capillary tubes), strings, nanoparticles (e.g. a carbon nanostructure such as a carbon nanotube, nanostring, nanoparticle, etc.), etc.

### Design, Production and Use of the Sensors

In some aspects, the sensor is prepared by thermal annealing of single-stranded DNA (ssDNA) oligonucleotides, such as the 5 strands discussed above. The oligonucleotides are typically designed manually to obtain the desired sensor. The hybridization length of ~20 base pairs and higher between the complementary strands gives a high stability of the sensors. Further, the GC content of about 50-60% is desirable when designing the sequences for high melting temperature (thermodynamic stability). The programs such as mFold or UNAFold are typically used to determine the melting temperature and change in free energy (ΔG) of the designed sequences, which are the parameters for stability. A biotin is incorporated in one of the strands to enable surface immobilization of sensors on the streptavidin-modified microscope slide via biotin/streptavidin interaction.

Methods of synthesizing oligonucleotides are well known in the art. To make a sensor, the oligonucleotides are suspended in a suitable buffer at a pH near neutrality, e.g. from about 7.0 to about 7.5. The concentrations of oligonucleotides that are employed are in the range of from about 0.5 to 5 µM, such as about a 1, 2, 3, 4, or 5 µM concentration for each oligonucleotide. The oligonucleotides are annealed by e.g. exposure to high temperatures (e.g. 85° C. or above, such as about 90 or 95° C.) for a short period of time to dissociate any base-paired strands. Then the mixture is slowly cooled e.g. to less than about 10° C., such as less than about 5° C., and annealing is allowed to proceed for e.g. at least about 1-4 hours, e.g. about 1, 2, 3, or 4 hours. An exemplary annealing protocol is to place the oligonucleotide mixture at 95° C. for 5 minutes, followed by a ramping down of the temperature to 4° C. during about 2 hours or less.

To use the sensor, a sample that comprises or is suspected of comprising a nucleic acid sequence of interest is contacted by the sensor, under conditions that permit binding of a nucleic acid sequence of interest to the single strand binding site of the sensor. The sequence of interest may be a mutated sequence or a wild-type sequence, and/or or a variant sequence. In some aspects, in order to detect a mutation, side-by-side preparation of aliquots of a sample are conducted using same sensor, one of which is to test for a mutant sequence and one of which is for detecting a corresponding non-mutant sequence (e.g. a positive control sequence) having a sequence identical to the mutant sequence, except that there is no mutation. If a mutation is present, one would expect a static signal (either due to no binding or formation of an incomplete junction as the mutated sequence is one of the core nucleotides at the four-way junction) from the sensor and a dynamic signal from the sensor in the non-mutant sample. Further, multiple sensors with different donor/acceptor distance may be employed in separate measurements to interrogate different sections of a single nucleic acid. Alternatively, multiple sensors may be used in side-by side preparations of samples to detect multiple (a plurality of) variants of a sequence, such as different virus variants in a single infected individual, or in multiple samples from multiple individuals. Of course, one or more positive controls can also be run in which binding of a nt sequence known to be fully complementary to the ss binding site of the sensor is detected (a dynamic signal), and/or one or more negative controls using nt sequences that are known to be non-complementary to the ss binding site of the sensor, for which only a static signal is detected.

While the sensors may be used to discriminate known mutations, the assessment of a nt sample believed to comprise a non-mutant sequence (and for which binding is thus expected) can yield a surprise result of no binding. The results can be confirmed, e.g. by sequencing of the nucleic acids in the sample and identification of the previously unknown or unsuspected mutant.

To bind to the sensor, targeted nucleic acids must be single-stranded. If the target has a few extra nucleotides (e.g. around 10 to 30 nucleotides) at the 3′ and 5′ ends, the sensor is expected to work normally. If the extra nucleotides at those ends are very large, they might prevent efficient binding to the sensor due to steric hindrance and prevent the junction from being dynamic due to the added weight etc. Thus, prior to analysis, it may be necessary to subject a sample to heat or other conditions sufficient to destroy base pairing between ds sequences, to remove proteins bound to the nucleic acids, and/or to destroy secondary and/or tertiary structure of the nucleic acid, that would otherwise interfere with hybridization. For example, long strands of DNA and RNA can be fragmented before applying the sample for detection. It is noted that all biomolecules typically exist in a dynamic flux of confirmations and bonding, so that some ds or otherwise occluded nucleic acids may be at least partially single stranded and not occluded, and hence detectable, even without heating or other pretreatment. Generally, a sample will at least be pretreated by concentrating; partial purification, e.g. by removal of proteins, carbohydrates, lipids; and optionally by amplification, etc. prior to analysis. Because the sensor exhibits femtomolar sensitivity, amplification is generally not required, but may be done if the user prefers.

The types of nucleic acids that can be detected by the sensor include both DNA and RNA. The nucleic acids may be of any type, for example, any DNA of interest such as DNA harboring single nucleotide polymorphisms (SNPs), various DNA biomarkers (e.g. those listed in the Table of Pharmacogenomic Biomarkers in Drug Labeling on the FDA website, DNA biomarkers of solid tumors (breast cancer, prostate cancer, etc.), and circulating DNAs, etc. RNA that may be detected includes microRNA (miRNA), small nuclear RNA (snRNA), Transfer-messenger RNA (tmRNA), Transfer RNA (tRNA), Signal recognition particle RNA (7SL RNA or SRP RNA), Ribosomal RNA (rRNA), Messenger RNA (mRNA), Small nuclear RNA (snRNA), Small nucleolar RNA (snoRNA), SmY RNA, SmY, Small Cajal body-specific RNA (scaRNA), Guide RNA (gRNA), Ribonuclease P (RNase P), Ribonuclease MRP (RNase MRP), Y RNA, Telomerase RNA Component, Spliced Leader RNA (SL RNA), Antisense RNA (aRNA, asRNA), Cis-natural antisense transcript (cis-NAT), CRISPR RNA (crRNA), Long noncoding RNA, MicroRNA (miRNA), Piwi-interacting RNA, Small interfering RNA (siRNA), Short hairpin RNA, Trans-acting siRNA, Repeat associated siRNA, Enhancer RNA (eRNA) Parasitic RNAs, Retrotransposons, Viral genome RNAs (Double-stranded RNA viruses, positive-sense RNA viruses, negative-sense RNA viruses, many satellite viruses and reverse transcribing viruses), Viroid RNA (Self-propagating in infected plants), Satellite RNA, Vault RNA (vRNA, vtRNA), etc.; nucleic acids of pathogenic organisms, for example Human Immunodeficiency viral DNA, coronavirus RNA (e.g. SARS COV-1 and 2, MERS, and variants thereof) etc.; alleles of genes in plants; etc.

The sensors may be used to discriminate any type of DNA mutation from its wild type sequence, e.g. one or more point mutations (substitutions, which can be a silent, missense or nonsense mutation), deletions or insertions. In some aspects, the one or more point mutations is/are a single nucleotide polymorphism (SNP) i.e. a substitution of a single nucleotide at a specific position in the genome, that is generally present in a sufficiently large fraction of the population (e.g. 1% or more). The SNP may be synonymous and nonsynonymous and may affect a coding region, gene splicing, transcription factor binding, messenger RNA degradation, or the sequence of noncoding RNA. SNPs play a direct role in disease by affecting, for example, a gene’s function, disease susceptibility, pathogenesis of disease (e.g. sickle-cell anemia, β-thalassemia, cystic fibrosis, risk for Alzheimer’s disease, cancer, infectious diseases (AIDS, leprosy, hepatitis, etc.), autoimmune diseases, neuropsychiatric diseases, etc. SNP’s can also inhibit or promote enzymatic activity, thereby impacting the efficacy of drugs and leading e.g. to increased or decreased rates of drug metabolism. Many SNPs are known (see, for example, the SNP database from the National Center for Biotechnology Information (NCBI), and the OMIM database which describes the association between polymorphisms and diseases), and any SNP can be detected in a subject using the sensors described herein.

The sensors are generally used to detect nucleotide sequences of interest in a sample, usually a biological sample from a subject. The subject may be any subject, archaea (single-celled organisms), plant or animal, that harbors a nucleic acid sequence of interest. The subjects may be, for example, mammals such as humans, companion pets, livestock, etc.; plants of any type; genetically engineered organisms that have been tagged with detectable and transmissible nucleic acids; bacteria or viruses; etc. suitable samples include but are not limited to: liquid samples such as blood, serum, saliva, amniotic fluid, urine, sap, etc.; a nucleic acid sample; an RNA transcript sample; an mRNA sample, DNA molecules, RNA and DNA isoform molecules; single nucleotide polymorphism molecules; or combinations thereof. For biological samples and/or extracts of any tissue or cells, nucleic acids have to be extracted and then placed in a suitable liquid carrier or solvent, e.g. physiologically compatible buffer, saline, etc. before analysis.

The sensors may be used in a variety of different fields or endeavors, including but not limited to: the early diagnosis of diseases including cancers; screening of genetic disorders (e.g. in an infant, child or adult) and/or for genetic counseling prior to pregnancy; in fetuses produced by in vitro fertilization prior to or after implantation; or in fetuses in utero resulting from natural pregnancies; in veterinary applications; in forensic applications; etc.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.)...”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### EXAMPLE 1. A Single-Molecule FRET-Based Dynamic DNA Sensor

Selective and sensitive detection of nucleic acid biomarkers is of great significance in early-stage diagnosis and targeted therapy. Therefore, the development of diagnostic methods capable of detecting diseases at the molecular level in biological fluids is vital to the emerging revolution in early diagnosis of diseases. However, most of the currently available ultrasensitive detection strategies involve either target/signal amplification or involve complex designs.

Over the years, DNA-based sensing using single molecule fluorescence resonance energy transfer (smFRET) has gained significant popularity due to its several advantages. First, sensors made up of DNA can be used to detect any DNA or RNA sequences using a hybridization approach, which offers a great deal of flexibility. Second, the donor/acceptor fluorophores can be directly incorporated into sensors to enable FRET so that the target does not need to be labeled. In this case the change in the FRET level after target binding can be used as a detection signal. Third, the smFRET approach provides quantitative information about the behavior of individual molecules, allowing simultaneous detection and quantitation. Additionally, the use of a total-internal reflection fluorescence (TIRF)-based FRET technique, as we have used in this study, enables high-throughput experiments by simultaneous imaging of several molecules in one movie.

In this Example, using a p53 tumor suppressor gene as a proof-of-concept target, whose mutation accounts for more than 50% of human cancers, we have demonstrated a simple background-free FRET-based sensor that enables an ultrasensitive detection of this biomarker. Unlike conventional fluorescence-based sensors, which require either a complex sensor design, signal amplification steps, or use of additional materials such as enzymes or nanocomposites, the sensor presented herein is simple and allows a single-step ultrasensitive detection of DNA biomarkers without target/signal amplification. Further, in contrast to other bulk FRET-based assays where detection relies on either increase or decrease in the FRET signal or fluorescence lifetime, the single-molecule approach used here takes advantage of the dynamic nature of a four-way DNA junction, which has several advantages. First, since the dynamic FRET is observed only in the presence of a target, this approach gives a zero background. In other words, there is no risk of false signal. Second, due to direct binding of target (no competition), our technique is ultrasensitive with a limit of detection of 50 fM (≈ 5 attomoles considering the sample volume of 100 µL) without the need for amplification and labeling. Third, the proposed method is direct because it does not require labeling of targets to achieve low fM detection. Fourth, unlike expensive enzymes or antibody-based sensors, this sensor can be readily prepared from short synthetic DNA strands and can be easily designed to detect “any” sequence of interest. Fifth, the sensor can discriminate targets even with single nucleotide mutations. Therefore, the proposed approach is novel and has the potential to benefit clinical practices by allowing a high-confidence early diagnosis of diseases.

### Results and Discussion

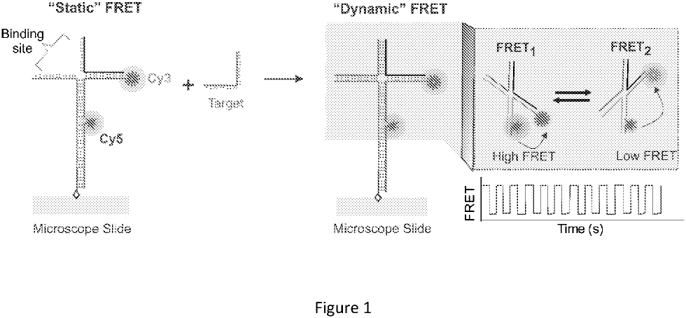

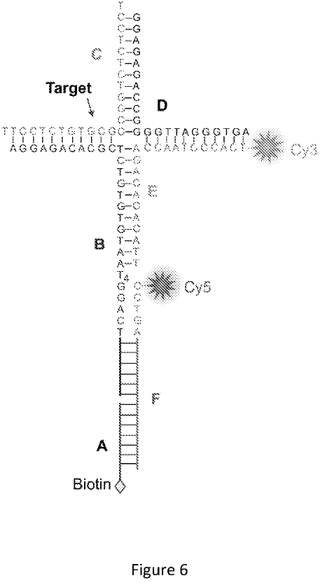

Sensor design and working principle. The sensor design and working principle is outlined in FIG. 1. The sensor molecules were custom designed using the nucleic acid hybridization principle. A retrospective design strategy was used to determine the single-stranded sequences that need to be incorporated into the binding regions of the sensor to enable target binding. In other words, when the target sequence is known, the target-binding region of the sensor can be created based on the principle of complementary base-pair hybridization. The rest of the sensor sequences were manually designed to obtain the desired sensor. A biotin was incorporated in one of the strands to allow surface immobilization of sensors on the streptavidin-modified microscope slide via biotin/streptavidin interaction (FIG. 1). In order to enable FRET, a donor (Cy3) and an acceptor (Cy5) fluorophore were incorporated into the sensor molecules using fluorophore-modified oligonucleotides. The detailed sequence and fluorophore-labeling scheme of the sensor is shown in FIG. 6.

In this study, we took advantage of the unique nature of the 4-way DNA junction (FIG. 1) that spontaneously interconverts between two stacked (X) conformers. To leverage this nature of the 4-way junction in sensing, the sensor was designed to have an incomplete junction so that it exhibits a medium but non-dynamic FRET level at the experimental acquisition time (50-100 ms) in the absence of target. However, binding of the target completes the 4-way junction resulting in a dynamic FRET. Therefore, a characteristic FRET pattern exhibiting fluctuations between a low- and a high-FRET state was expected as a detection signal.

Single-molecule sensing. First, the sensor molecules were immobilized on the microscope slide using the biotin/streptavidin interaction (FIG. 1) as described in Methods. To remove the unbound sensor molecules, an imaging buffer containing an oxygen scavenger system (OSS) was injected and incubated for ~2 min to let the OSS equilibrate. The OSS was used to retard fluorophore blinking and photobleaching upon laser illumination. The fluorescence movies were collected using a total internal reflection fluorescence (TIRF) microscope. Briefly, the flow cell was then irradiated with a 532 nm laser to excite the donor (Cy3) fluorophore of the surface-tethered molecules to enable fluorescence resonance energy transfer (FRET). The fluorescence emissions of both Cy3 and Cy5 fluorophores were recorded at 10 frames per second (100 ms time resolution and camera gain of 200). The presence of a Cy5 fluorophore was confirmed by direct excitation using a red laser (639 nm) toward the end of the movies. The movies were processed with IDL and MATLAB codes (see Methods) to create intensity-time traces.^{38,44,45} Only those single molecules showing evidence for the presence of both donor/acceptor fluorophores and single-step photobleaching of fluorophores were selected for further analysis.

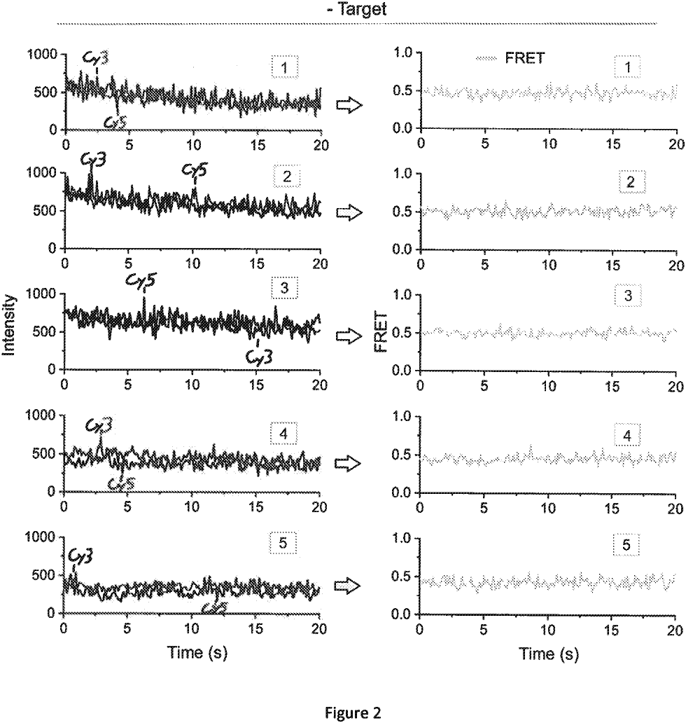

We first examined the sensor alone in the absence of the target by analyzing intensity-time traces and the corresponding FRET state. Some typical molecules from this experiment are shown in FIG. 2. As expected, the donor/acceptor emission traces were relatively flat without any definite dynamic pattern (FIG. 2). When the raw intensity traces were converted to FRET traces (see Methods for detail), all the molecules show relatively static FRET-time traces with a FRET value of ~0.5. These experiments suggested that the sensor molecules behave uniformly.

Interestingly, when the experiment in FIG. 2 was repeated after injection of target (p53 tumor suppressor gene: 5´-TTCCTCTGTGCGCCGGTCTCTCCT, SEQ ID NO: 13) and incubated for ~20 min, we observed a very different behavior of sensor molecules. In the presence of the target, sensor molecules showed very clear dynamics of Cy3 and Cy5 fluorescence intensities that were anti-correlated. The typical single molecule traces from this experiment are shown in FIG. 3. When the raw intensity-time traces were converted to FRET-time traces, all of the dynamic molecules showed a very clear switching pattern between ~0.3 and ~0.7 FRET states (FIG. 3). Such dynamics is an inherent behavior of the fully formed 4-way junction and it is important to note that such dynamic switching was absent in the target-free experiments (FIG. 2). Therefore, this sensing approach demonstrated a high-confidence detection of target. Inspired by these results, we next performed experiments at various concentrations of the target to determine the analytical sensitivity of the sensor.

Determining the analytical sensitivity. To determine the analytical sensitivity of the sensor, a series of experiments were performed at various concentrations of target. The percentage of dynamic molecules (≈detection) under each concentration of target was then determined by dividing the number of dynamic molecules by the total number of single molecules (see Methods for details). Control experiments were performed in the absence of target, which yielded no dynamic molecules. Through single-molecule counting, we determined that there were ~2% dynamic molecules (4 out of 204 total molecules) at 50 femtomolar (fM) target and no dynamic molecules were observed below this concentration. Therefore, the limit of detection (LOD) of this method was determined to be 50 fM under our experimental conditions. When the percentage of dynamic molecules was plotted against the target concentration (FIG. 4), it showed a linearity up to around 10 pM target (FIG. 4 inset), after which it was curved and eventually plateaued. It is important to notice that there were no dynamic molecules in the absence of target (FIG. 2), and thus this sensing approach exhibits a zero-background. Further, given the flow cell volume of ∼100 µL, the detection limit of 50 fM translates to 5 attomoles, which means that this sensing method is highly sensitive. In addition, it has a large dynamic range (~3 orders of magnitude) extending to ∼100 pM.

From the stability standpoint, DNA-based sensors that are made up of short synthetic DNA strands can have stability issues if they must be stored for a long period of time (weeks). Since the sensor used in this study has short arms (11 bp each) and we observed a loss of Cy3 signal after about a week after sensor assembly, we sought to increase the arm length of the sensor and test it for dynamics and LOD. For this, we prepared a construct with slightly longer D/E and B/C arms (increased by 2 and 1 bp respectively) and extended the Strand E by 4As to complement 4T in Strand B (Table 1 and FIG. 6). Apart from these changes, the revised construct was identical to the original design. We tested this revised design for five different concentrations of targets (50 fM, 100 fM, 200 pM, 300 pM and 800 pM) and obtained similar results as in the original construct in terms of fraction of dynamic molecules. These results showed that the design can be tuned to enhance the sensor stability without compromising the sensitivity of the sensor. Given that most sensing approaches available today require either amplification of target (enzymatic or non-enzymatic), labeling of target, or some sort of signal amplification such as the use of nanomaterials or nanocomposites to reach low nM to fM detection limits, the sensing approach that we demonstrated here offers an ultrasensitive detection of nucleic acids in a simpler format.

TABLE 1

DNA oligonucleotides used in this study

| Strand Name | Sequence (5´-3´) |
|---|---|
| Strand A- (Biotin labeled) | /biotin/-AC GCG CTG GGC TAC GTC TTG CTG GCC GCA T (SEQ ID NO: 1) |
| Strand B | CTG TGC GGT ATT TCA CAC CGT TAG CTC AGG TTT TAA TGT GTG TCT CGC ACA GAG GA (SEQ ID NO: 2) |
| Strand C (p53gene-T1) | TTC CTC TGT GCG CCG GTC TCT CCT (SEQ ID NO: 3) |
| Strand D | GGA GAG ACC GGG GTT AGG GTG A (SEQ ID NO: 4) |
| Strand E (Cy3 strand) | /5Cy3/TCA CCC TAA CCA GAC ACA CAT T (SEQ ID NO: 5) |
| Strand F (Cy5 Strand) | /Cy5/CCT GAG CTA ACG GTG TGA AAT ACC GCA CAG ATG CGG CCA GCA AGA CGT AGC CCA GCG CGT (SEQ ID NO: 6) |
| Strand C (Mut1) | TTC CTC TGT GCT CCG GTC TCT CCT (SEQ ID NO: 7) |
| Strand C (Mut2) | TTC CTC TGT GCA CCG GTC TCT CCT (SEQ ID NO: 8) |
| Strand C (Mut3) | TTC CTC TCT GCG GCG GTC TCT CCT (SEQ ID NO: 9) |
| Modified sequence for better stability (added nucleotides are in bold) of the sensor | |
| Strand B′ | CTG TGC GGT ATT TCA CAC CGT TAG CTC AGG TTT TAA TGT GTG TCT CGC ACA GAG GAA (SEQ ID NO: 10) |
| Strand D′ | GGA GAG ACC GGG GTT AGG GTG CGA (SEQ ID NO: 11) |
| Strand E′ | /5Cy3/TCG CAC CCT AAC CAG ACA CAC ATT AAA A (SEQ ID NO: 12) |

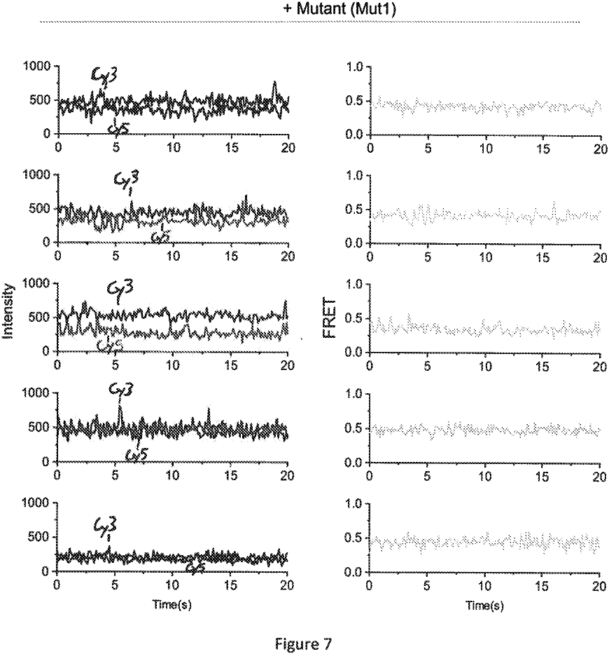

In addition to sensitivity, another requirement of a sensor is its specificity. Therefore, to test the specificity of the sensor, we designed three mutant sequences and compared the results with the original p53 target. As shown in FIG. 5A, mutants 1 and 2 have their 12 ^{th} nucleotide altered from the 5′-end. The rationale for this design was that, since the dynamic FRET is the result of an intact four-way junction, a single mismatch at the vicinity of the junction could result in loss in dynamics so that the signal in the presence of mutant will not overlap with the one from the specific target. Interestingly, both mutants showed less than 2% dynamic-like molecules even at nearly saturating concentration of mutants (100 pM). The typical single-molecule traces involving mutant 1 are shown in the Supplementary FIG. 7. We also tested another mutant with two mutation sites (one on each arm) and obtained similar results as seen for single mutants 1 and 2. Overall, these experiments demonstrated that the 4-way-junction based sensing can easily discriminate a fully matched target from its single-nucleotide mismatch mutants. In other words, these sensors can be retrospectively designed such that the mutation site directly falls at the junction to fully discriminate the mutant from the target.

Given that the body responds to onset of diseases by the altered release of certain molecules such as miRNAs or hormones, sensors that are compatible with biological fluids warrant a wider range of applications. Serum is a suitable biological fluid for this purpose, therefore, we employed human serum and tested the performance of the sensor at 0, 10 and 100 pM of target and directly compared the results obtained in a regular 1x Tris buffer. Interestingly, the percentage of dynamic molecules determined in serum (10%) and regular buffer were the same within the error (FIG. 5B). Further, similar to the regular buffer result, there were no dynamic molecules detected in the absence of target in serum, demonstrating that the sensor offers a background-free detection in serum.

### Conclusions

We have developed a novel single-step fluorescence-based sensor to detect DNA biomarkers, which we demonstrated using a p53 tumor suppressor gene as a proof-of-concept target. Since the detection relies on target-induced formation of dynamic molecules, this sensing strategy enables a background-free, ultrasensitive, and high-confidence detection of DNA without the need for target/signal amplification. Further, a LOD of ~5 attomoles can be achieved without labeling the target. The sensor design is comprised of 4-way junction with a 22-nucleotides binding site (11 nucleotides on each arm), which is a perfect size to implement for miRNA detection as the average mature miRNAs size falls between 20-23 nts. Further, the detection is based on the direct hybridization of sequences, which is a great advantage as the sensor can be easily designed to detect any sequence of interest by simply swapping the two unlabeled DNA strands. In addition, the LOD of 50 fM (~5 attomoles) is in the range of typical nucleic acid biomarkers including miRNAs, pathogenic DNA and circulating tumor DNAs in biological samples and thus this sensor offers direct applications in biotechnology to detect, for example, trace amounts of nucleic acid biomarkers and pathogenic DNAs.

### Methods

Chemical reagents and DNA sequences. Biotinylated bovine serum albumin (bBSA) was purchased from Thermo Scientific. It was dissolved in filtered sterile water at 1 mg/mL and stored at -20° C. until needed. The reagents for the oxygen scavenging system including protocatechuate 3,4-dioxygenase (PCD), 6-hydroxy-2,5,7,8-tetramethylchroman-2-carboxylic acid (Trolox), tris(hydroxymethyl)-aminomethane (Tris), ethylenediaminetetraacetic acid disodium salt (EDTA), and acetic acid were also purchased from Fisher Scientific. Protocatechuic acid (PCA) and streptavidin were purchased from VWR. All DNA sequences were purchased from Integrated DNA Technologies (IDT Inc.) and stored at -20° C. until needed. Biotinylated and fluorophore-labeled sequences were purchased HPLC-purified.

Sensor Design and Preparation. The sensor was prepared by thermal annealing of five single-stranded DNA (ssDNA) oligonucleotides at 1 µM concentrations in 1×TAE-Mg buffer (40 mM Tris, 20 mM acetic acid, 1 mM EDTA and 100 mM Mg²⁺, pH 7.4). Among the five oligonucleotides, two were modified with either a Cy3 or Cy5 fluorophore to enable FRET and another strand was modified with a biotin so that sensors could be immobilized on the microscope slide via biotin/streptavidin interaction. The sample was heated at 95° C. for 5 minutes and then the temperature was gradually ramped down to 4° C. in the duration of <2 hours. A donor (Cy3) and an acceptor (Cy5) fluorophore were introduced using labeled ssDNA to incorporate a FRET pair in each molecule.

Single-molecule fluorescence imaging. The sensor molecules were immobilized on the surface of a quartz slide flow cell functionalized with biotin-BSA and streptavidin as described elsewhere.^{44,45} Briefly, after mounting the flow cell on the microscope stage, a 60-80 pM sensor solution prepared in 1× TAE buffer consisting of 100 mM MgCl2 and an oxygen scavenging system (OSS: 4 mM Trolox, 10 mM PCA, 100 nM PCD) was injected into the flow cell and incubated for ~30 seconds to allow surface immobilization of sensor molecules via biotin/streptavidin interaction. The unbound molecules were then removed by flushing the flow cell with 400 µL of imaging buffer (OSS containing 1×TAE buffer spiked with 100 mM Mg²⁺, pH 7.4).

The fluorescence imaging was carried out using a prism-based total internal reflection fluorescence (pTIRF) microscope in a 1×TAE buffer spiked with 100 mM Mg²⁺ (pH 7.4). Using a 532 nm laser, the Cy3 fluorophores were continuously excited while emissions from Cy3 and Cy5 fluorophores were simultaneously recorded through green and red channels (512 × 256 pixels) on an EMCCD camera at a 100 ms time resolution. In all sensing experiments, a target DNA solution of a given concentration was injected into the flow cell and incubated for ~20 minutes before fluorescence imaging. The imaging was performed at room temperature (23° C.).

Single-molecule data acquisition and analysis. The single-molecule movies were processed using IDL and MATLAB scripts and fluorescence-time trajectories of individual molecules were obtained as described previously. The presence of an active FRET-pair was confirmed by turning on a 639 nm laser to directly excite the Cy5 fluorophore towards the end of each movie. Only those molecules that show the presence of both fluorophores and a single step photobleaching were selected for further data analysis. The FRET efficiency value was calculated using the equation: I_{A}/(I_{D} + I_{A}), where I_{A} and I_{D} represent the background-corrected fluorescence intensities of the acceptor and donor fluorophores, respectively. The dynamic vs static molecules were assigned by manual counting of two types of molecules as the dynamics was overwhelmingly clear on the FRET-time traces exhibiting many transitions between the FRET levels of ~0.3 and ~0.7. A standard curve was prepared by plotting the percentage of dynamic molecules at different concentrations of target DNA. This calculation was performed by dividing the number of dynamic molecules by the total number of selected single molecules. Standard deviation of the percentage of dynamic molecules was determined using at least three groups of independent movie files at each concentration of target.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A sensor, comprising a substrate, and
an incomplete 4-way DNA junction immobilized on the substrate;
wherein the incomplete 4-way DNA junction comprises
a first arm of double-stranded(ds) DNA;
a second arm of dsDNA;
a third arm of single-stranded DNA;
a fourth arm of single-stranded DNA;
a fluorescence resonance energy transfer (FRET) donor; and
a FRET acceptor,
wherein the ssDNA of the third are and the fourth arm form a single strand binding site complementary to a targeted nucleic acid sequence; and
wherein one of the FRET donor and the FRET acceptor is attached to the dsDNA of the first arm and the other of the FRET donor and the FRET acceptor is attached to dsDNA of the second arm of the sensor.

2. The sensor of claim 1, wherein
the FRET donor and the FRET acceptor exhibit a detectable static mid-FRET state when the targeted nucleic acid sequence is not bound to the sensor; and
the FRET donor and the FRET acceptor undergo detectable continuous dynamic switching between a low- FRET state and high-FRET state when the targeted nucleic acid sequence is bound to the sensor.

3. The sensor of claim 1, wherein the incomplete 4-way DNA junction is converted to a complete 4-way DNA junction when the targeted nucleic acid sequence is bound to the sensor.

4. The sensor of claim 1, wherein the incomplete 4-way DNA junction is immobilized on the substrate via a biotin/streptavidin interaction.

5. A method of detecting a targeted nucleic acid sequence in a biological sample, comprising
i) contacting the biological sample with the sensor of claim 1; and
ii) detecting continuous dynamic switching of the FRET donor and the FRET acceptor between low-FRET and high-FRET levels, wherein detection of continuous dynamic switching indicates that the targeted nucleic acid sequence is bound to the sensor.

6. The method of claim 5, wherein the targeted nucleic acid sequence comprises at least one mutation.

7. The method of claim 6, wherein the at least one mutation is a point mutation, a deletion or an insertion.

8. The method of claim 7, wherein the point mutation is a single nucleotide polymorphism (SNP).


SEQUENCE LISTING (13 sequences)

SEQ ID NO: 1 - DNA, 30 bp, Artificial Sequence
  Synthetic oligonucleotide
acgcgctgggctacgtcttgctggccgcat

SEQ ID NO: 2 - DNA, 56 bp, Artificial Sequence
  Synthetic oligonucleotide
ctgtgcggtatttcacaccgttagctcaggttttaatgtgtgtctcgcacagagga

SEQ ID NO: 3 - DNA, 24 bp, Artificial Sequence
  Synthetic oligonucleotide
ttcctctgtgcgccggtctctcct

SEQ ID NO: 4 - DNA, 22 bp, Artificial Sequence
  Synthetic oligonucleotide
ggagagaccggggttagggtga

SEQ ID NO: 5 - DNA, 22 bp, Artificial Sequence
  Synthetic oligonucleotide
tcaccctaaccagacacacatt

SEQ ID NO: 6 - DNA, 60 bp, Artificial Sequence
  Synthetic oligonucleotide
cctgagctaacggtgtgaaataccgcacagatgcggccagcaagacgtagcccagcgcgt

SEQ ID NO: 7 - DNA, 24 bp, Artificial Sequence
  Synthetic oligonucleotide
ttcctctgtgctccggtctctcct

SEQ ID NO: 8 - DNA, 24 bp, Artificial Sequence
  Synthetic oligonucleotide
ttcctctgtgcaccggtctctcct

SEQ ID NO: 9 - DNA, 24 bp, Artificial Sequence
  Synthetic oligonucleotide
ttcctctctgcggcggtctctcct

SEQ ID NO: 10 - DNA, 57 bp, Artificial Sequence
  Synthetic oligonucleotide
ctgtgcggtatttcacaccgttagctcaggttttaatgtgtgtctcgcacagaggaa

SEQ ID NO: 11 - DNA, 24 bp, Artificial Sequence
  Synthetic oligonucleotide
ggagagaccggggttagggtgcga

SEQ ID NO: 12 - DNA, 28 bp, Artificial Sequence
  Synthetic oligonucleotide
tcgcaccctaaccagacacacattaaaa

SEQ ID NO: 13 - DNA, 24 bp, Artificial Sequence
  Synthetic supressor
ttcctctgtgcgccggtctctcct